(19) United States
(12) Patent Application Publication
(10) Pub. No.: US 2025/0228961 A1
(43) Pub. Date: Jul. 17, 2025

(54) PRACTICAL GLYCAN ENGINEERING METHOD FOR THE PREPARATION OF HOMOGENOUS ANTIBODY CONJUGATES

(71) Applicant: Wisconsin Alumni Research Foundation, Madison, WI (US)

(72) Inventors: Weiping Tang, Middleton, WI (US); Deqin Cai, Madison, WI (US); Yuan Zhao, Madison, WI (US)

(73) Assignee: Wisconsin Alumni Research Foundation, Madison, WI (US)

(21) Appl. No.: 19/015,033

(22) Filed: Jan. 9, 2025

Related U.S. Application Data
(60) Provisional application No. 63/620,047, filed on Jan. 11, 2024.

(51) Int. Cl. A61K 47/68; C07H 19/24

(52) U.S. Cl. CPC A61K 47/6889; C07H 19/24

(57) ABSTRACT

An acetal- or ketal-containing disaccharide that is useful as a linker in antibody-drug conjugation. The disaccharide comprises a hexose linked to a N-acetylglucosamine oxazoline, and further comprises an acetal or ketal moiety bonded at a 4-position hydroxyl oxygen atom and a 6-position hydroxyl oxygen atom of the hexose. The acetal or ketal group can bear a variety of functional groups. Also provided are an antibody drug conjugate comprising an antibody linked to a therapeutic drug via the disaccharide. Also provided is a two-step method of making the acetal- or ketal-containing disaccharide.

### CROSS-REFERENCE TO RELATED APPLICATIONS

Priority is hereby claimed to provisional application Ser. No. 63/620,047, filed Jan. 11, 2024, which is incorporated herein by reference.

### FEDERAL FUNDING STATEMENT

This invention was made with government support under GM148266 awarded by the National Institutes of Health. The government has certain rights in the invention.

### BACKGROUND

Antibody drug conjugates (ADCs) have become one of the most effective modalities for the treatment of cancers. These molecules use antibodies as delivery vehicles to target a therapeutic molecule to a tumor. Because ADCs are tumor-targeted therapeutics, they display fewer side effects than traditional chemotherapies. Most ADCs on the market or in clinical trials exploit conjugation methods for native antibodies with chemically activatable side chains (e.g. lysine, cysteine, glutamine) or glycans. (See Hu et al. “Towards the Next Generation of Biomedicines by Site-Selective Conjugation” Chem. Soc. Rev. 2016, 45, 1691; Yamada et al. “Recent Chemical Approaches for Site-Specific Conjugation of Native Antibodies: Technologies toward Next-Generation Antibody-Drug Conjugates” Chembiochem 2019, 20, 2729; Khongorzul et al. “Antibody-Drug Conjugates: A Comprehensive Review” Mol. Cancer Res. 2020, 18, 3; Gauzy-Lazo et al. “Advances in Antibody-Drug Conjugate Design: Current Clinical Landscape and Future Innovations” Slas Discovery 2020, 25, 843; Li and Wang. “Chemoenzymatic Methods for the Synthesis of Glycoproteins” Chem. Rev. 2018, 118, 8359; Walsh et al. “Site-Selective Modification Strategies in Antibody-Drug Conjugates” Chem. Soc. Rev. 2021, 50, 1305.)

One challenge in using glycans as the attachment points is the diversity of the glycans on the antibody. The native antibodies produced from nature all have diverse glycans at the end of the oligosaccharides. This diversity of glycans leads to heterogenic mixtures of products. This heterogeneity affects the pharmacokinetics of the ADC. Any technical improvement on the glycan engineering method may have impact for the development of all antibody drug conjugates.

A number of methods have been explored to control the site and number of linker/drug conjugated to the antibody, with the aim of producing more homogenous ADCs:

Lysine modification. On average, an IgG has 80 lysine residues, and more than 20 of these are accessible to solvents (McCombs et al. “Antibody Drug Conjugates: Design and Selection of Linker, Payload and Conjugation Chemistry” AAPS J. 2015, 17, 339). Random acylation of the lysine residues on antibodies can often lead to issues such as decreased stability and aggregation (Wakankar et al. “Physicochemical Stability of the Antibody-Drug Conjugate Trastuzumab-Dm1: Changes Due to Modification and Conjugation Processes” Bioconjugate Chem. 2010, 21, 1588). The variability and heterogeneity of the products are also not desirable for further therapeutic development.

Cysteine modification. On IgG1, there are 16 pairs of cysteine residues, with 12 pairs being intra-chain and 4 pairs forming inter-chain disulfide bonds. The solvent-accessible inter-chain cysteines have been utilized for preparing ADCs. (See McCombs et al. “Antibody Drug Conjugates: Design and Selection of Linker, Payload and Conjugation Chemistry” AAPS J. 2015, 17, 339.) The re-bridging of the inter-chain S—S bond can improve the homogeneity and stability of the ADCs (Bryden et al. “Regioselective and Stoichiometrically Controlled Conjugation of Photodynamic Sensitizers to a Her2 Targeting Antibody Fragment” Bioconjugate Chem. 2014, 25, 611; Badescu et al. “Bridging Disulfides for Stable and Defined Antibody Drug Conjugates” Bioconjugate Chem. 2014, 25, 1124). The ligand/antibody ratio can be controlled at 4.

Glutamine modification. A chemo-enzymatic method for the attachment of ligands to a glutamine residue 295 on each of the two heavy chains through a MTGase-catalyzed acyl transfer reaction has also been reported. (See Jeger et al. “Site-Specific and Stoichiometric Modification of Antibodies by Bacterial Transglutaminase” Angew. Chem. Int. Ed. 2010, 49, 9995; Dennler et al. “Transglutaminase-Based Chemo-Enzymatic Conjugation Approach Yields Homogeneous Antibody-Drug Conjugates” Bioconjugate Chem. 2014, 25, 569; Schneider et al. “Recent Progress in Transglutaminase-Mediated Assembly of Antibody-Drug Conjugates” Anal. Biochem. 2020, 595; Anami and Tsuchikama. Transglutaminase-Mediated Conjugations. In Antibody-Drug Conjugates: Methods and Protocols; Tumey, L. N., Ed.; 2020; Vol. 2078, pp 71; Hussain et al. “Toward Homogenous Antibody Drug Conjugates Using Enzyme-Based Conjugation Approaches” Pharmaceuticals 2021, 14.) The glycan on Asn-297 needs to be removed by PNGase F for an efficient acyl-transfer reaction. Several ADCs prepared using this method have entered clinical trials, though they lack any glycan. (See Strop et al. “Rn927c, a Site-Specific Trop-2 Antibody-Drug Conjugate (Adc) with Enhanced Stability, Is Highly Efficacious in Preclinical Solid Tumor Models” Mol. Cancer Ther. 2016, 15, 2698; King et al. “A Phase 1, Dose-Escalation Study of Pf-06664178, an Anti-Trop-2/Aur0101 Antibody-Drug Conjugate in Patients with Advanced or Metastatic Solid Tumors” Invest. New Drugs 2018, 36, 836.)

Glycan modification. Although cysteine modification offers more precision than lysine labelling, the resulting products are still heterogeneous mixtures due to the existence of different glycans on the two Asn-297 residues in the Fc domain. Completely removing the glycan on Asn-297 by PNGase F followed by MTGase treatment can provide homogenous antibody conjugates, some of which have entered clinical trials. However, concerns about lower drug antibody ratio (DAR) and the complete absence of glycans, which may reduce the solubility of the antibody, have led many researchers exploring the glycan engineering method, which also has the potential to provide homogenous antibody conjugates. In addition, it allows the optimization of the antibody by introducing different glycans bearing a diverse range of functional groups. (See Wang et al. Glycoengineering of Antibodies for Modulating Functions. In Annual Review of Biochemistry, Vol 88; Kornberg, R. D., Ed.; 2019; Vol. 88, pp 433; Boune et al. “Principles of N-Linked Glycosylation Variations of Igg-Based Therapeutics: Pharmacokinetic and Functional Considerations” Antibodies 2020, 9.)

The discovery of endoglycosidases Endo-S, Endo-S2, and their mutants allowed the preparation of a homogenous antibody glycoform (Wang et al. Glycoengineering of Antibodies for Modulating Functions. In Annual Review of Biochemistry, Vol 88; Kornberg, R. D., Ed.; 2019; Vol. 88, pp 433; Huang et al., “Chemoenzymatic Glycoengineering of Intact Igg Antibodies for Gain of Functions” J. Am. Chem. Soc. 2012, 134, 12308; Li et al. “Glycosynthase Mutants of Endoglycosidase S2 Show Potent Transglycosylation Activity and Remarkably Relaxed Substrate Specificity for Antibody Glycosylation Remodeling” J. Biol. Chem. 2016, 291, 16508; Tang et al. “Chemoenzymatic Synthesis of Glycoengineered Igg Antibodies and Glycosite-Specific Antibody-Drug Conjugates” Nat. Protoc. 2017, 12, 1702). These enzymes can convert diverse glycans on antibody 4 to product 5 bearing a GlcNAc residue with or without the Fuc-residue (FIG. 1). Product 5 can then be used as the substrate for a series of glycosyl transferases to attach tetrasaccharides or more complex sugars with an oxazoline motif to the antibody to form product 7 (Wang et al. Glycoengineering of Antibodies for Modulating Functions. In Annual Review of Biochemistry, Vol 88; Kornberg, R. D., Ed.; 2019; Vol. 88, pp 433; Boune et al. “Principles of N-Linked Glycosylation Variations of Igg-Based Therapeutics: Pharmacokinetic and Functional Considerations” Antibodies 2020, 9). However, the most practical way to prepare the complex glycosyl donors is the extraction of N-glycans from large amount of natural sources such as eggs and soybean flour. (See Li et al. “Chemoenzymatic Methods for the Synthesis of Glycoproteins” Chem. Rev. 2018, 118, 8359.) These complex natural N-glycans need to be further functionalized for drug conjugation, which is not trivial. Nevertheless, this method is used due to the lack of better options.

It was recently found that a series of simple disaccharide oxazoline derivatives (e.g., 3, FIG. 1) bearing diverse functional groups could be used as glycosyl donors for naturally occurring wild type Endo-S2 enzyme-catalyzed antibody glyco-engineering (Zhang et al. “General and Robust Chemoenzymatic Method for Glycan-Mediated Site-Specific Labeling and Conjugation of Antibodies: Facile Synthesis of Homogeneous Antibody-Drug Conjugates” ACS Chem. Biol. 2021, 16, 2502; Zhang et al. “Synthesis and Evaluation of Three Azide-Modified Disaccharide Oxazolines as Enzyme Substrates for Single-Step Fc Glycan-Mediated Antibody-Drug Conjugation” Bioconjugate Chem. 2022, 33, 1179). Most importantly, product 4 is resistant to Endo-S2 mediated hydrolysis due to the truncated nature of the product compared to the substrate. The de-glycosylation and transglycosylation could be done in one-pot highly efficiently using catalytic amount of Endo-S2 enzyme at neutral pH and room temperature (Zhang et al. “General and Robust Chemoenzymatic Method for Glycan-Mediated Site-Specific Labeling and Conjugation of Antibodies: Facile Synthesis of Homogeneous Antibody-Drug Conjugates” ACS Chem. Biol. 2021, 16, 2502). For example, antibody conjugates 3A, 3B, or 3C were used as the glycosyl donor to introduce 2, 4, and 6 units of azide to the antibody. In addition, the mannose in the disaccharide glycosyl donor could also be replaced by glucose (e.g., 6A) or galactose (e.g., 6B). (See Zhang et al. “Synthesis and Evaluation of Three Azide-Modified Disaccharide Oxazolines as Enzyme Substrates for Single-Step Fc Glycan-Mediated Antibody-Drug Conjugation” Bioconjugate Chem. 2022, 33, 1179; Shi et al. “One-Step Synthesis of Site-Specific Antibody-Drug Conjugates by Reprograming Igg Glycoengineering with Lacnac-Based Substrates” Acta Pharm. Sin. B. 2022, 12, 2417.) However, the synthesis of the above disaccharides with one or more handles generally takes ten steps or more. Recently, a method involving chemoenzymatic oxidation followed by chemical modification was reported for the synthesis of glycosyl donors from commercially available N-acetyllactosamine (LacNAc) (Shi et al. “One-Step Synthesis of Site-Specific Antibody-Drug Conjugates by Reprograming IgG Glycoengineering with LacNAc-Based Substrates” Acta Pharmaceutica Sinica B 2022, 12, 2417). However, the enzyme is not readily available, and the oxidation product is not very stable.

The present disclosure addresses limitations of current methods and develops a practical glycan engineering method for the preparation of homogenous antibody conjugates.

### SUMMARY

Antibody drug conjugates (ADCs) have become one of the most effective modalities for the treatment of cancers. These molecules use antibodies as delivery vehicles to target a therapeutic molecule to a tumor. Because ADCs are tumor-targeted therapeutics, they display fewer side effects than traditional chemotherapies. Most ADCs on the market or in clinical trials exploit conjugation methods for native antibodies with chemically activatable side chains (e.g. lysine, cysteine, glutamine) or glycans. One challenge in using glycans as the attachment points is the diversity of the glycans on the antibody. This diversity of glycans leads to heterogenic mixtures of products. This heterogeneity affects the pharmacokinetics of the ADC.

Endo-S2 and mutants of this enzyme have been developed to hydrolyze the complex glycan chains on antibodies to ones bearing a GlcNAc residue. The sugars can then be used as the substrate for a series of glycosyl transferases to attach tetrasaccharides or more complex sugars with an oxazoline motif to the antibody. However, the chemistry required to form one of these disaccharide linkers requires 10 or more steps. This leads to poor manufacturing outcomes and mixed products.

The present disclosure develops a simple, two-step process for creating a disaccharide handle useful as a linker in antibody-drug conjugation. It is recognized that the Endo-S2 enzyme used to hydrolyze glycan chains from antibodies yields a functional group that can react with an oxazoline to create the linker. An exemplary acetal-containing disaccharide is created via a two-step chemical synthesis using the commercially available LacNAc. The linker appears stable in the experimentation conducted. This is a platform that can be used for any antibody-drug conjugate. The synthetic method for making the molecule is simpler than previously reported methods for making sugar linkers.

Thus, disclosed herein is a disaccharide comprising a hexose linked to a N-acetylglucosamine oxazoline, and further including an acetal or ketal moiety bonded at a 4-position hydroxyl oxygen atom and a 6-position hydroxyl oxygen atom of the hexose. The hexose is selected from the group consisting of galactose, glucose, and mannose. In certain versions, the hexose is galactose.

The acetal or ketal moiety of the disaccharide can bear a variety of functional groups. In certain versions, the acetal or ketal moiety bears a therapeutic drug to form an antibody drug conjugate via the disaccharide linker.

Also provided herein is an antibody drug conjugate comprising an antibody linked to a therapeutic drug via the disaccharide linker disclosed herein, wherein the N-acetylglucosamine oxazoline of the disaccharide is linked to the antibody and the acetal or ketal moiety of the disaccharide bears the therapeutic drug.

Also provided herein is a method of making an acetal- or ketal-containing disaccharide, comprising: a) reacting a disaccharide comprising a hexose linked to a N-acetylglucosamine with a compound selected from an aldehyde, a ketone, and an acetal, to yield a product containing an acetal or ketal moiety bonded at a 4-position hydroxyl oxygen atom and a 6-position hydroxyl oxygen atom of the hexose; and b) convert the product of step a) to an oxazoline.

The hexose is selected from the group consisting of galactose, glucose, and mannose. In certain versions, the hexose is galactose. In certain versions, step b) comprises treating the product of step (a) with 2-chloro-1,3-dimethylimidazolinium chloride to yield the oxazoline.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1. Conjugation of a tag to antibody by the Endo-S2-catalyzed chemo-enzymatic method.

FIG. 2. Conjugation of an acetal-containing tag to antibody by the Endo-S2-catalyzed chemo-enzymatic method.

FIG. 3. MALDI-LC-MS detection of deglycosylation and transglycosylation of Herceptin. A) intact antibody. B) Deglycosylation of Herceptin using 1% Endo-S2. C) Transglycosylation of Herceptin using 1% Endo-S2.

FIG. 4. Optimization of the Endo-S2 amount used for deglycosylation and transglycosylation of Herceptin. A) intact antibody. B) Deglycosylation of Herceptin using 0.1% Endo-S2. C) Transglycosylation of Herceptin using 0.1% Endo-S2. D) Deglycosylation of Herceptin using 0.2% Endo-S2. E) Transglycosylation of Herceptin using 0.2% Endo-S2. F) Deglycosylation of Herceptin using 0.5% Endo-S2. G) Transglycosylation of Herceptin using 0.5% Endo-S2.

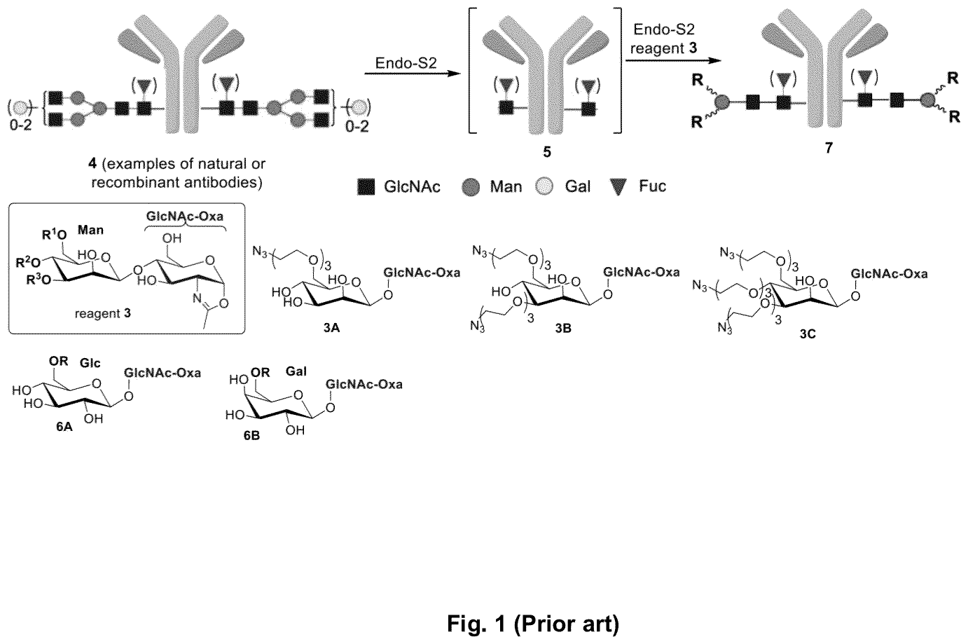

### DETAILED DESCRIPTION

### Abbreviations and Definitions

ADCs: Antibody drug conjugates; DAR: Drug antibody ratio;
GlcNAc: N-Acetylglucosamine; LacNAc: N-Acetyllactosamine;
MTGase: Microbial transglutaminase;
PNGase F: Peptide-N4-(N-acetyl-beta-glucosaminyl) asparagine amidase F.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present disclosure shall include the corresponding plural characteristic or limitation, and vice-versa, unless otherwise specified or clearly implied to the contrary by the context in which the reference is made.

As used herein, the term “or” is an inclusive “or” operator and is equivalent to the term “and/or” unless the context clearly dictates otherwise.

The elements and method steps described herein can be used in any combination whether explicitly described or not, unless otherwise specified or clearly implied to the contrary by the context in which the referenced combination is made.

All combinations of method or process steps as used herein can be performed in any order, unless otherwise specified or clearly implied to the contrary by the context in which the referenced combination is made.

The compositions and methods of the present disclosure can comprise, consist of, or consist essentially of the essential elements and limitations of the composition and method described herein, as well as any additional or optional ingredients, components, or limitations described herein or otherwise useful in synthetic organic chemistry. The disclosure provided herein may be practiced in the absence of any element or step which is not specifically disclosed herein.

It is understood that the disclosure is not confined to the particular elements and method steps herein illustrated and described, but embraces such modified forms thereof as come within the scope of the claims.

### The Acetal-Containing Disaccharide

Disclosed herein is a disaccharide with an acetal or ketal moiety that is useful as a linker in antibody-drug conjugation. The disaccharide comprises a hexose linked to a GlcNAc oxazoline, and further includes an acetal or ketal moiety bonded at a 4-position hydroxyl oxygen atom and a 6-position hydroxyl oxygen atom of the hexose. The acetal or ketal moiety can bear diverse functional groups (e.g., a cytotoxic drug or other therapeutic or diagnostic agent that is useful in cancer treatment).

The hexose in the disaccharide may be galactose, glucose, or mannose. In certain versions, the hexose is galactose. The disaccharide can be derived from LacNAc and has the following structures:

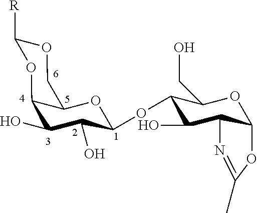

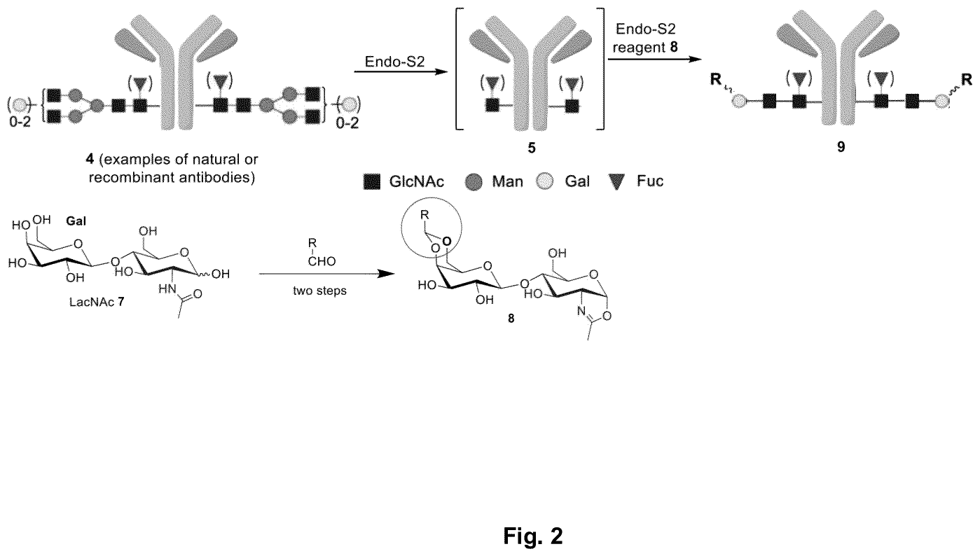

The acetal-containing disaccharide derived from LacNAc
   (Compound 8 in FIG. 2; acetal moiety circled)

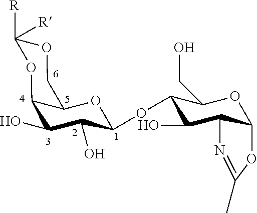

The ketal-containing disaccharide derived from LacNAc

As shown in FIG. 2, the glycosyl donor 8 can be efficiently attached to an antibody to form antibody conjugate 9 using naturally occurring wild type Endo-S2 enzyme.

This method is by far the most practical and efficient way to prepare homogenous antibody conjugates, one of the biggest classes of therapeutics. The central innovation is the use of the disaccharide with an acetal or ketal moiety bonded at a 4-position hydroxyl oxygen atom and a 6-position hydroxyl oxygen atom of the hexose part for antibody conjugation. R and R′ can be virtually any pharmacological agent, without limitation.

### Method of Making

The acetal- or ketal-containing disaccharides disclosed herein can be made through a simple, two-step process. As shown in Scheme 1, using LacNAc as an exemplary starting material, the first step involves adding an acetal group bonded at the 4-position hydroxyl oxygen atom and the 6-position hydroxyl oxygen atom of the galactose part of LacNAc, by reacting LacNAc with an aldehyde or an acetal bearing a functional group R. The second step involves converting the product of the first step to an oxazoline, e.g., by treatment with DMC (2-chloro-1,3-dimethylimidazolinium chloride). The Example section describes in detail syntheses of exemplary compounds DC02Lac-1 and DC02Lac-2 by reacting LacNAc with an aldehyde and an acetal, respectively. As shown in Scheme 2, the ketal-containing disaccharides can be made through a similar pathway, using LacNAc as an exemplary starting material.

### Scheme 1: Two-Step Process to Synthesize the Acetal-Containing Disaccharide

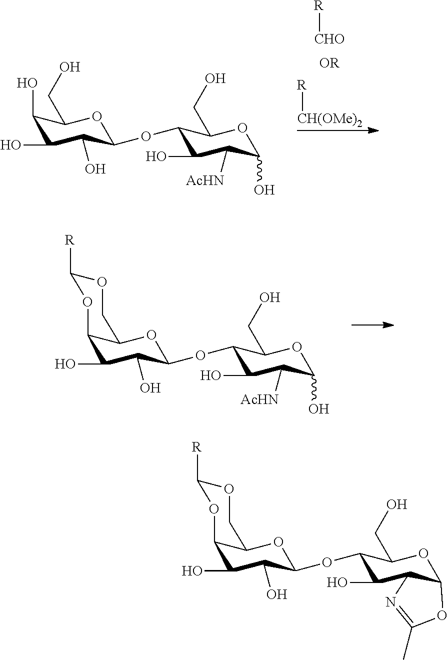

### Scheme 2: Two-Step Process to Synthesize the Ketal-Containing Disaccharide

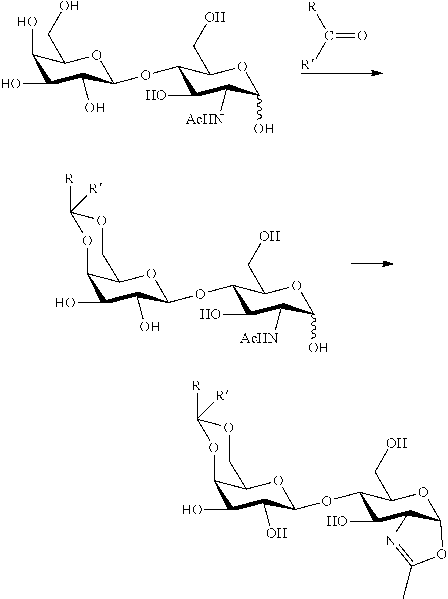

### EXAMPLES

### Example 1

### Synthesis of DC02Lac-1

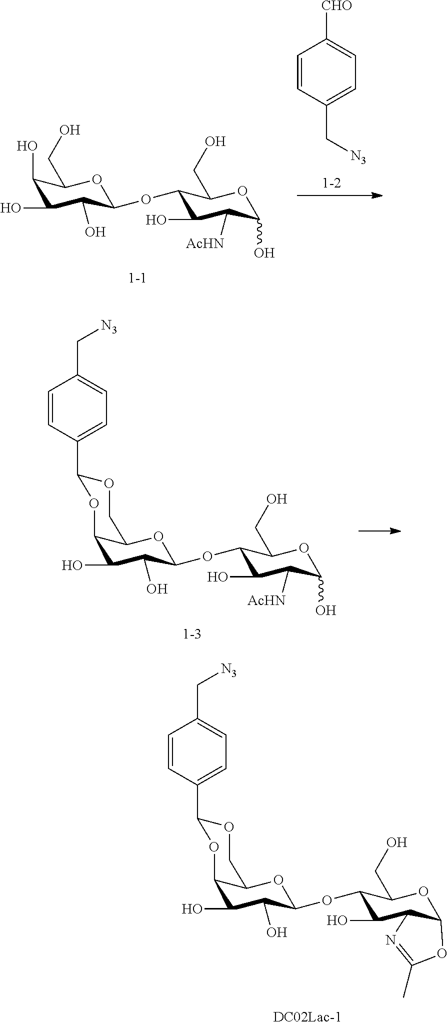

### Step 1:

CSA (5.4 mg, 0.023 mmol, 0.3 e.q.) was added to a mixture of LacNAc 1-1 (30.0 mg, 0.078 mmol, 1.0 e.q.) and 1-2 (63.1 mg, 0.39 mmol, 5.0 e.q.) in DMSO (1 mL). The mixture was then co-distilled with toluene at 50° C. for several times until the LCMS indicated majority of 1-1 was reacted. The reaction was quenched by one drop of triethylamine. The result mixture was then purified by a preparative C-18 column. The column was eluted with a 5%-95% gradient of aqueous acetonitrile containing 0.1% formic acid to afford 1-3 (12.0 mg, yield 29.1%). ¹H NMR (400 MHZ, MeOD) δ 7.60-7.54 (m, 2H), 7.38-7.33 (m, 2H), 5.64 (s, 1H), 5.11 (d, J=2.8 Hz, 0.8H, α-H^{GlcNAc}), 4.60-4.56 (m, 0.2H, β-H^{GlcNAc}) 4.49 (dt, J=5.3, 1.6 Hz, 1H), 4.36 (s, 2H), 4.25-4.12 (m, 3H), 3.99-3.87 (m, 4H), 3.84-3.76 (m, 1H), 3.70-3.59 (m, 4H), 1.98 (d, J=2.1 Hz, 3H). ¹³C NMR (100 MHZ, MeOD) δ 172.2, 138.2, 136.3, 127.7, 126.6, 103.6, 100.5, 90.9, 79.9, 76.0, 72.1, 70.5, 70.1, 69.3, 68.8, 66.9, 60.4, 54.2, 53.8, 21.2. HRMS (ESI) for C₂₂H₃₀N₄NaO₁₁ ([M+Na⁺]): calcd 549.1809, found 549.1800.

### Step 2:

DMC (2-chloro-1,3-dimethylimidazolinium chloride, 18.6 mg, 0.11 mmol, 10.0 e.q.) was added to 1-3 (6 mg, 0.011 mmol, 1.0 e.q.) and K₃PO₄ (46.7 mg, 0.22 mmol, 20 e.q.) in D₂O (1 mL) in ice-bath. The reaction mixture was then stirred at RT overnight. The reaction was subjected to HNMR directly to determine the end of reaction. After the reaction was finished, the result mixture was purified by G-10 column to afford DC02Lac-1 (3.9 mg, yield 67.3%). 1H NMR (⁴⁰⁰ MHZ, D₂O) δ 7.56-7.49 (m, 2H), 7.43-7.36 (m, 2H), 6.03 (dd, J=7.3, 4.5 Hz, 1H), 5.69 (s, 1H), 4.49 (d, J=7.9 Hz, 1H), 4.46 (dd, J=3.3, 1.8 Hz, 1H), 4.39 (s, 2H), 4.30 (d, J=3.4 Hz, 1H), 4.20 (t, J=1.9 Hz, 2H), 4.16-4.11 (m, 1H), 3.80-3.70 (m, 3H), 3.69-3.56 (m, 3H), 3.41 (ddd, J=8.8, 6.3, 2.6 Hz, 1H), 2.00 (d, J=1.9 Hz, 3H). ¹³C NMR (100 MHz, DMSO) δ 165.8, 138.9, 136.3, 128.5, 127.1, 105.7, 100.0, 99.8, 80.5, 72.4, 71.3, 70.5, 70.0, 69.2, 68.1, 66.6, 62.0, 53.8, 14.0. HRMS (ESI) for C₂₂H₂₈KN₄O₁₀ ([M+K⁺]): calcd 547.1442, found 547.1433.

### Example 2

### Synthesis of DC02Lac-2

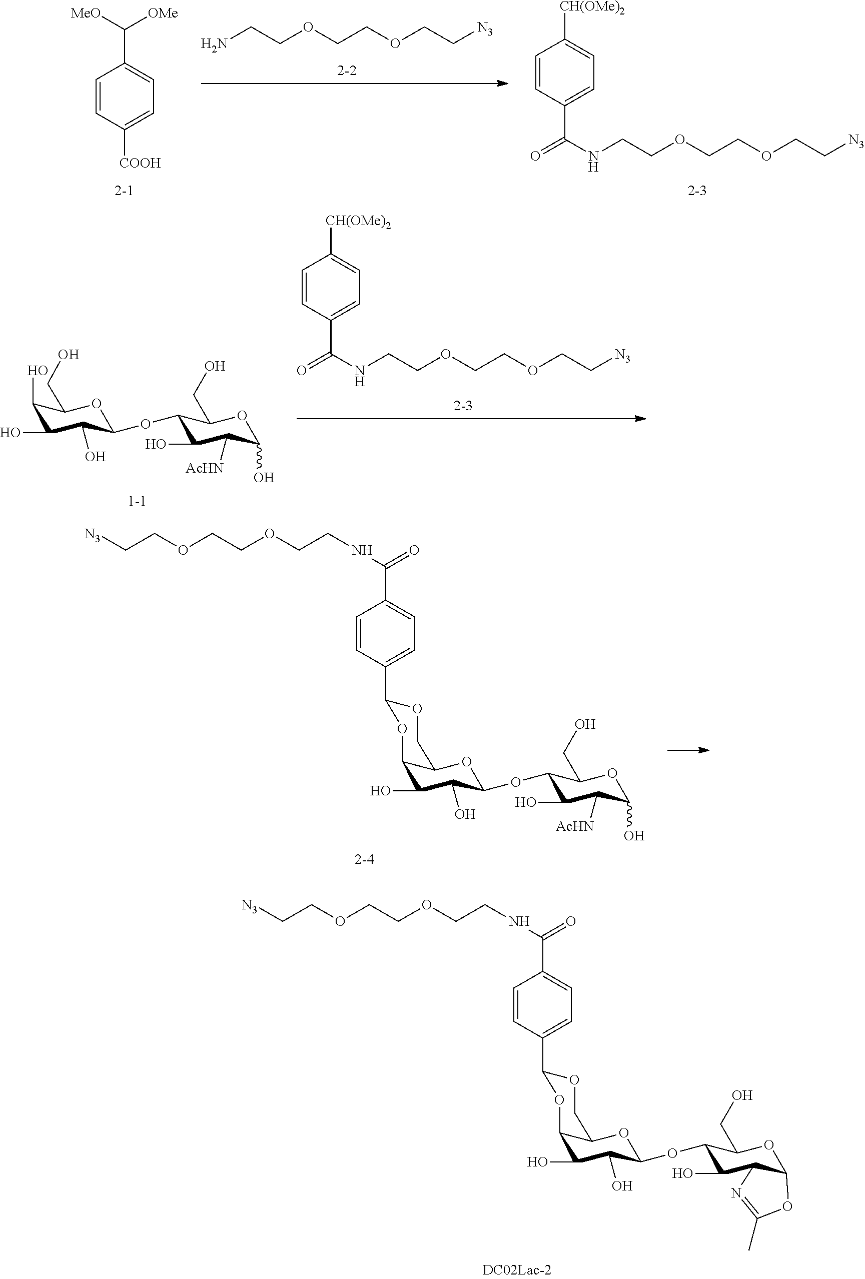

### Step 1:

EDCI (42 mg, 0.22 mmol, 1.22 e.q.) was added to a mixture of 2-1 (40 mg, 0.2 mmol, 1.1 e.q.), 2-2 (31 mg, 0.0.18 mmol, 1.0 e.q.), DMAP (2.4 mg, 0.02 mmol, 0.11 e.q.) and triethylamine (30.4 μL, 0.22 mmol, 1.22 e.q.) in DCM (2 mL). The reaction was diluted with DCM after LCMS indicated the end of the reaction. The mixture was washed with saturated sodium bicarbonate aqueous solution and water. And dried over sodium sulfate. The result mixture was purified by flash column with ethyl acetate/n-hexane as eluents to afford 2-3 (59 mg, yield 94.1%) as oil. A minor part of acetal was hydrolyzed into aldehyde during column purification. This will not affect the next step. ¹H NMR (400 MHZ, CDCl₃) δ 7.97 (s, 1H), 7.84-7.78 (m, 2H), 7.58-7.51 (m, 2H), 5.46 (s, 1H), 3.73-3.67 (d, J=3.4 Hz, 10H), 3.39 (t, J=5.0 Hz, 2H), 3.35 (s, 6H).

### Step 2:

CSA (2.7 mg, 0.012 mmol, 0.3 e.q.) was added to a mixture of LacNAc 1-1 (15.0 mg, 0.039 mmol, 1.0 e.q.) and 2-3 (20.4 mg, 0.058 mmol, 1.5 e.q.) in DMSO (1 mL). The mixture was then co-distilled with toluene at 50° C. for several times until the LCMS indicated majority of 1-1 was reacted. The reaction was quenched by one drop of triethylamine. The result mixture was then purified by a preparative C-18 column. The column was eluted with a 5%-95% gradient of aqueous acetonitrile containing 0.1% formic acid to afford 2-4 (4.0 mg, yield 15.2%). ¹H NMR (400 MHZ, D₂O) δ 7.76-7.71 (m, 2H), 7.63-7.56 (m, 2H), 5.74 (s, 1H), 5.14 (d, J=3.0 Hz, 0.81H, α-H^{GlcNAc}), 4.51 (d, J=7.9 Hz, 1H), 4.32 (d, J=3.6 Hz, 1H), 4.25-4.14 (m, 2H), 3.97-3.73 (m, 6H), 3.73-3.58 (m, 11H), 3.54 (t, J=5.3 Hz, 3H), 3.40-3.29 (m, 2H), 1.96 (s, 3H). ¹³C NMR (100 MHZ, D₂O) δ 174.7, 170.6, 140.3, 134.7, 127.3, 126.6, 103.0, 100.2, 90.5, 79.2, 75.7, 74.8, 71.2, 70.4, 70.3, 69.5, 69.5, 69.2, 69.1, 69.0, 68.8, 66.6, 53.7, 50.1, 39.6, 21.8. HRMS (ESI) for C₂₈H₄₁N₅NaO₁₄ ([M+Na⁺]): calcd 694.2548, found 694.2533.

### Step 3:

DMC (2-chloro-1,3-dimethylimidazolinium chloride, 10.1 mg, 0.06 mmol, 10.0 e.q.) was added to 2-4 (4 mg, 0.006 mmol, 1.0 e.q.) and K₃PO₄ (25.4 mg, 0.12 mmol, 20 e.q.) in D₂O (1 mL) in ice-bath. The reaction mixture was then stirred at RT overnight. The reaction was subjected to HNMR directly to determine the end of reaction. After the reaction was finished, the result mixture was purified by G-10 column to afford DC02Lac-2 (2.8 mg, yield 71.8%). ¹H NMR (400 MHZ, D₂O) δ 7.80-7.69 (m, 2H), 7.64-7.57 (m, 2H), 6.03 (d, J=7.2 Hz, 1H), 5.73 (s, 1H), 4.49 (d, J=7.9 Hz, 1H), 4.45 (dd, J=3.2, 1.7 Hz, 1H), 4.31 (d, J=3.7 Hz, 1H), 4.26-4.17 (m, 2H), 4.14 (ddd, J=7.3, 3.3, 1.7 Hz, 1H), 3.80-3.58 (m, 14H), 3.55 (q, J=5.7 Hz, 2H), 3.40 (ddd, J=8.8, 6.2, 2.5 Hz, 1H), 3.35 (dd, J=5.6, 4.2 Hz, 2H), 2.00 (d, J=1.8 Hz, 3H). ¹³C NMR (125 MHz, DMSO) δ 169.0, 166.2, 138.3, 132.7, 125.3, 124.6, 102.6, 98.3, 97.8, 76.7, 73.7, 69.2, 68.9, 68.3, 67.5, 67.2, 67.0, 66.8, 66.5, 64.5, 63.0, 59.7, 48.0, 37.5, 10.9. HRMS (ESI) for C₂₈H₃₉N₅NaO₁₃ ([M+Na⁺]): calcd 676.2442, found 676.2429.

### Example 3

### Preparation of Azido-Functionalized Antibody

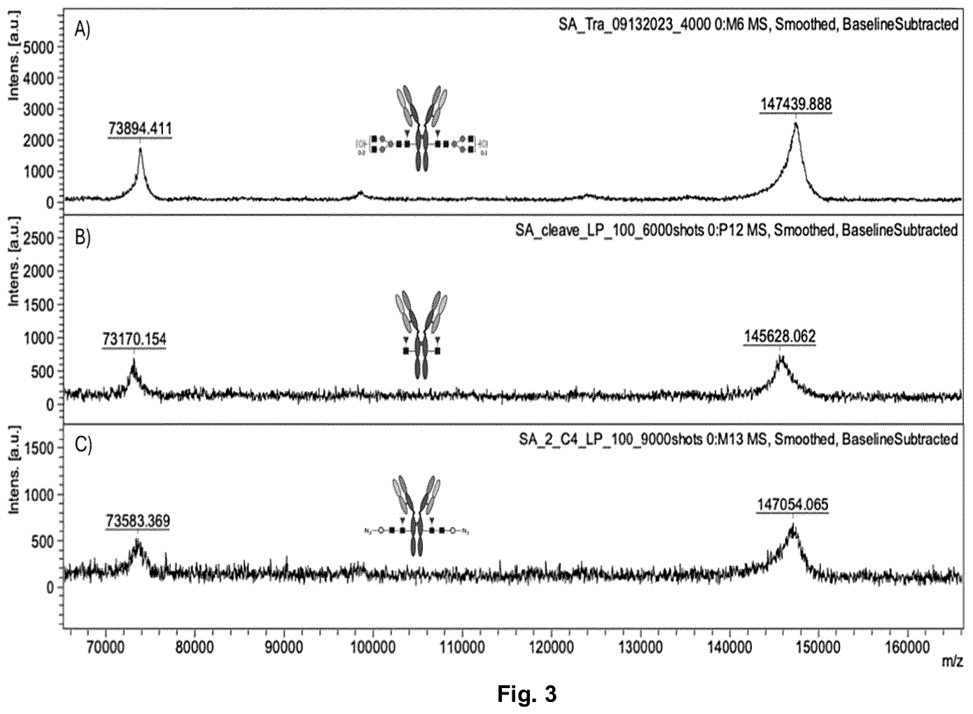

10 mg/ml Herceptin was incubated with 80 eq lac compound bearing an azide group and Endo-S2 (1% w/w) overnight at room temperature in 20 μl PBS buffer. The next day, the deglycosylation and transglycosylation of Herceptin were confirmed with MALDI-LC-MS (FIG. 3). As shown in the figure, the azide group was successfully conjugated to Herceptin using the disaccharide linker.

### Optimization of the Endo-S2 Amount Used for Preparation Azido Functionalized Antibody

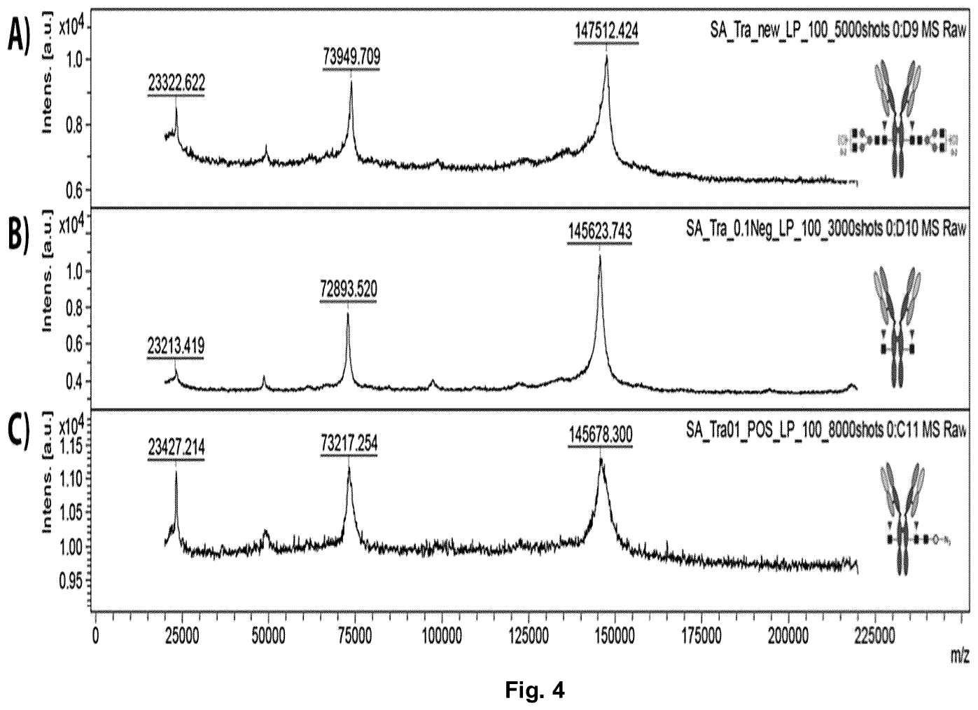

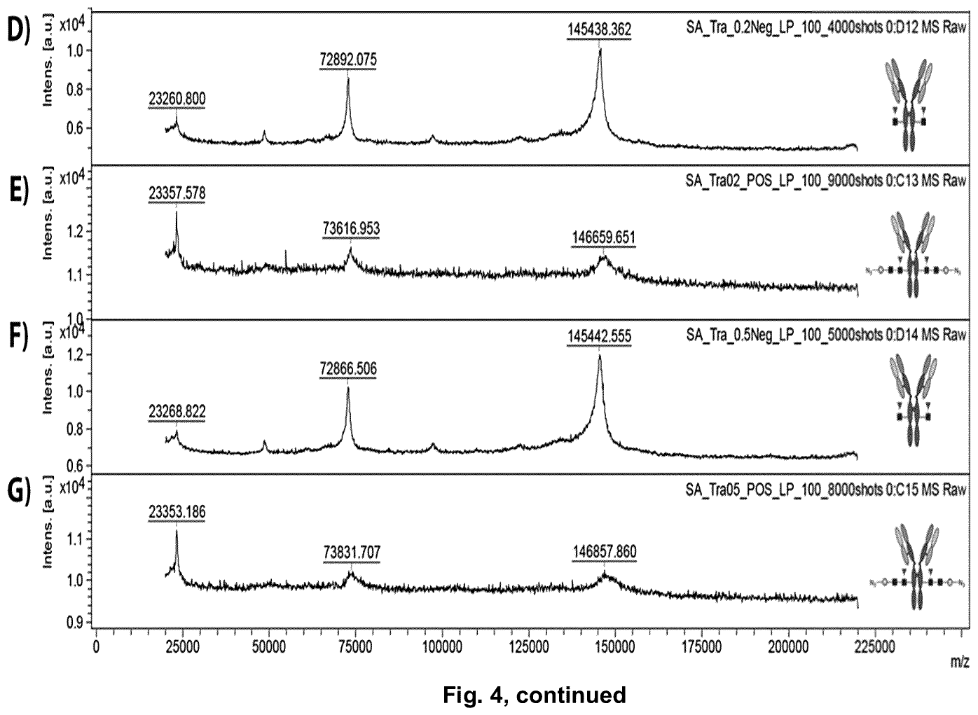

10 mg/ml Herceptin was incubated with 80 eq lac compound and different amount of Endo-S2 (0.1%, 0.2%, and 0.5%) overnight at room temperature in 20 μl PBS buffer. The next day, the deglycosylation and transglycosylation of Herceptin were confirmed with MALDI-LC-MS (FIG. 4). As shown in the figure, partial conjugation was achieved with 0.1% Endo-S2, while complete conjugation was observed at 0.2% and 0.5%, respectively.

What is claimed is:

1. A disaccharide comprising a hexose linked to a N-acetylglucosamine oxazoline, and further including an acetal or ketal moiety bonded at a 4-position hydroxyl oxygen atom and a 6-position hydroxyl oxygen atom of the hexose.

2. The disaccharide of claim 1, wherein the hexose is selected from the group consisting of galactose, glucose, and mannose.

3. The disaccharide of claim 1, wherein the hexose is galactose.

4. The disaccharide of claim 1, wherein the acetal or ketal moiety bears a therapeutic drug.

5. An antibody drug conjugate comprising an antibody linked to a therapeutic drug via the disaccharide of claim 1, wherein the N-acetylglucosamine oxazoline of the disaccharide is linked to the antibody and the acetal or ketal moiety of the disaccharide bears the therapeutic drug.

6. A method of making an acetal- or ketal-containing disaccharide, comprising:
a) reacting a disaccharide comprising a hexose linked to a N-acetylglucosamine with a compound selected from an aldehyde, a ketone, and an acetal, to yield a product containing an acetal or ketal moiety bonded at a 4-position hydroxyl oxygen atom and a 6-position hydroxyl oxygen atom of the hexose; and
b) converting the product of step a) to an oxazoline.

7. The method of claim 6, wherein the hexose is selected from the group consisting of galactose, glucose, and mannose.

8. The method of claim 6, wherein the hexose is galactose.

9. The method of claim 6, wherein step b) comprises treating the product of step (a) with 2-chloro-1,3-dimethylimidazolinium chloride to yield the oxazoline.
